(19) United States
(12) Patent Application Publication
(10) Pub. No.: US 2024/0164741 A1
(43) Pub. Date: May 23, 2024

(54) DIAPHRAGMATIC ULTRASONOGRAPHY IMAGE ALIGNMENT AND MOTION COMPENSATION FOR CONTRALATERAL COMPARISON

(71) Applicant: KONINKLIJKE PHILIPS N.V., EINDHOVEN (NL)

(72) Inventors: Rafael Wiemker, Hamburg (DE); Roberto Buizza, Eindhoven (NL); Joerg Sabczynski, Hamburg (DE); Jaap Roger Haartsen, Eindhoven (NL); Thomas Koehler, Hamburg (DE); Cornelis Petrus Hendriks, Eindhoven (NL); Michael Polkey, London (GB); Nataly Wieberneit, Hamburg (DE)

(21) Appl. No.: 18/233,511

(22) Filed: Aug. 14, 2023

Related U.S. Application Data
(60) Provisional application No. 63/426,931, filed on Nov. 21, 2022.

(51) Int. Cl. A61B 8/08; A61B 8/00

(52) U.S. Cl. CPC A61B 8/08; A61B 8/463; A61B 8/469; A61B 8/5246; A61B 8/5276; A61B 8/5284; A61B 8/54

(57) ABSTRACT

A respiration monitoring device for monitoring a respiratory system of a patient includes an electronic controller configured to receive first ultrasound imaging data as a function of time acquired of a first portion of the respiratory system of the patient over a first time interval; receive second ultrasound imaging data as a function of time acquired of a second portion of the respiratory system of the patient over a second time interval wherein the second portion of the respiratory system of the patient is contralateral to the first portion of the respiratory system of the patient; and present a graphical user interface (GUI) displaying a visualization including simultaneous display of an image of the first portion of the respiratory system of the patient and an image of the second portion of the respiratory system of the patient respectively.

### CROSS-REFERENCE TO RELATED APPLICATIONS

This patent application claims the priority benefit under 35 U.S.C. § 119(e) of U.S. Provisional Application No. 63/426,931, filed on Nov. 21, 2022, the contents of which are herein incorporated by reference.

The following relates generally to the respiratory therapy arts, mechanical ventilation arts, ventilator induced lung injury (VILI) prevention arts, ultrasound imaging arts, and related arts.

### BACKGROUND

Diaphragmatic ultrasonography (US) allows for quantification of diaphragm thickness, strain (rate) and excursion, and with this also the respiratory rate and duration of each contraction. Diaphragm thickness (expressed as thickening fraction) and strain reflect contractile activity and correlate well with diaphragmatic electrical activity and diaphragmatic pressure. Consequently, thickness and strain may be used as a surrogate for respiratory effort. Applications of diaphragmatic ultrasound include assessment of diaphragm function, atrophy detection, weaning prediction, and mechanical ventilation (MV) setting management. Other applications are asynchrony detection and proportional ventilation (non-invasive neurally adjusted ventilatory assist (NAVA)). The use of diaphragmatic ultrasound in mechanical ventilation is gaining attention and therefore, technical problems and use cases are currently being investigated.

A diaphragm thickening fraction (TFdi or TFDI) as measured by ultrasound (US) is carried out by an operator who looks at the patient and takes an ultrasound image at end inhalation and end exhalation. The diaphragm thickness fraction is typically determined by subtracting the end inhalation thickness Tₑᵢ from the end exhalation thickness Tₑₑ and dividing the difference by the exhale thickness according to Equation 1:

$$TFdi = \frac{{T}_{ei} - {T}_{ee}}{{T}_{ee}} * 100 \% \qquad ( 1 )$$

The thickness of the diaphragm as varying over the breathing cycle (or more precisely the thickening during inspiration) is a surrogate for the patient's respiratory effort (see, e.g., Tuinman P R, Jonkman A H, Dres M, Shi Z H, Goligher E C, Goffi A, de Korte C, Demoule A, Heunks L. Respiratory muscle ultrasonography: methodology, basic and advanced principles and clinical applications in ICU and ED patients—a narrative review. Intensive Care Med. 2020 April; 46(4):594-605. doi: 10.1007/s00134-019-05892-8. Epub 2020 Jan. 14. PMID: 31938825; PMCID: PMC7103016).

A diaphragm excursion (Edi) as well as the absolute thickening (Tdi) or fractional thickening (TFdi) as measured using a hand-held ultrasound probe are widely recognized parameters to assess diaphragm function, and to optimize ventilator support and weaning, as well as to assess and optimize the effect of artificial stimulation of the thoracic diaphragm using phrenic nerve stimulation (PNS) by implanted electric leads or non-invasively. The diaphragm thickening depends on diaphragmatic activity and reflects the diaphragmatic work of breathing (WoB) (i.e., the respiratory effort).

Diaphragmatic excursion as well as diaphragmatic thickness are both functions over the respiratory cycle, and independent clinical variables. Contra-lateral asymmetry (i.e., between left and right lung) may be an indication of medical complications (e.g., regional pulmonary tissue stiffness, regional diaphragmatic atrophy, phrenic nerve damage, etc.).

While diaphragmatic US with a handheld probe is widely practiced, the visual appraisal of the moving US image by the medical observer is confounded by certain complications. For example, excursion and thickening are two parameters which are difficult to appraise separately, because both change simultaneously, and thickness variation may be only a tenth of the motion amplitude. The diaphragm is constantly moving within the field of view (FOV), either being passively pushed by the mechanical ventilation, or actively driven by muscular activity. In another example, contralateral symmetry, or more general regional symmetries, are difficult to appraise visually with a single US probe, as typically the FOV of a hand-held US-probe covers only a part of either one of the left or right lung area and a corresponding portion of the extended diaphragm muscle. In another example, the effects of MV and phrenic nerve stimulation (PNS), respectively, as well as their gradual settings, are difficult to distinguish as both are supposed to cause a similar respiratory motion, however by different mechanisms. During the weaning process, the passive MV-caused part is desired to be reduced in favor of muscular motion, but the appearance differences may be subtle.

The following discloses certain improvements to overcome these problems and others.

### SUMMARY

In one aspect, a respiration monitoring device for monitoring a respiratory system of a patient, in which the respiratory system comprising a left lung, a right lung, and a thoracic diaphragm of the patient, includes an electronic controller configured to receive first ultrasound imaging data as a function of time acquired of a first portion of the respiratory system of the patient over a first time interval; receive second ultrasound imaging data as a function of time acquired of a second portion of the respiratory system of the patient over a second time interval wherein the second portion of the respiratory system of the patient is contralateral to the first portion of the respiratory system of the patient; and present a graphical user interface (GUI) displaying a visualization including simultaneous display of an image of the first portion of the respiratory system of the patient and an image of the second portion of the respiratory system of the patient respectively generated from the first ultrasound imaging data and second ultrasound imaging data.

In another aspect, a respiration monitoring method for monitoring a respiratory system of a patient, in which the respiratory system comprising a left lung, a right lung, and a thoracic diaphragm of the patient, includes receiving first ultrasound imaging data as a function of time acquired of a first portion of the respiratory system of the patient over a first time interval; receiving second ultrasound imaging data as a function of time acquired of a second portion of the respiratory system of the patient over a second time interval wherein the second portion of the respiratory system of the patient is contralateral to the first portion of the respiratory system of the patient; and presenting a GUI displaying a visualization including simultaneous display of an image of the first portion of the respiratory system of the patient and an image of the second portion of the respiratory system of the patient respectively generated from the first ultrasound imaging data and second ultrasound imaging data.

One advantage resides in providing an intuitive and time-synchronized contralateral ultrasound-based visualization of left and right lungs and/or corresponding thoracic diaphragm muscle portions.

Another advantage resides in determining and visualizing contra-lateral asymmetry between matching organs (i.e., lungs) in a patient.

Another advantage resides in providing time-synchronized visualization of both the left and right lung (and/or corresponding diaphragm) portions using a user-selected synchronization signal, thereby providing intuitive visualization of information such as the extent to which different synchronization signals correlate with the dynamic motion of the lungs.

Another advantage resides in providing contra-lateral ultrasound visualization of a bilaterally symmetric organ or organ system such as the lungs or heart using only a single ultrasound probe with limited depth-of-penetration.

Another advantage resides in providing such a contra-lateral ultrasound visualization with a single ultrasound probe facilitating visual comparison of functioning of the left and right portions of the bilaterally symmetric organ or organ system.

Another advantage resides in distinguishing between effects on a patient from mechanical ventilation and phrenic nerve stimulation.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically shows a simplified representation of the left and right lungs and surrounding tissue.

FIG. 2 diagrammatically shows an illustrative respiration monitoring device in accordance with the present disclosure.

FIGS. 3A and 3B show example flow charts of operations suitably performed by the device of FIG. 1.

FIGS. 4 and 5 show different embodiments of image processing performed by the device of FIG. 1.

FIG. 6 diagrammatically shows a time-synchronized contralateral ultrasound-based visualization of left and right thoracic diaphragm muscle portions, along with the corresponding synchronization signal and a user dialog for selecting the synchronization signal.

### DETAILED DESCRIPTION

As used herein, the singular form of “a,” “an,” and “the” include plural references unless the context clearly dictates otherwise. As used herein, statements that two or more parts or components are “coupled,” “connected,” or “engaged” shall mean that the parts are joined, operate, or co-act together either directly or indirectly, i.e., through one or more intermediate parts or components, so long as a link occurs. Directional phrases used herein, such as, for example and without limitation, top, bottom, left, right, upper, lower, front, back, and derivatives thereof, relate to the orientation of the elements shown in the drawings and are not limiting upon the scope of the claimed invention unless expressly recited therein. The word “comprising” or “including” does not exclude the presence of elements or steps other than those described herein and/or listed in a claim. In a device comprised of several means, several of these means may be embodied by one and the same item of hardware.

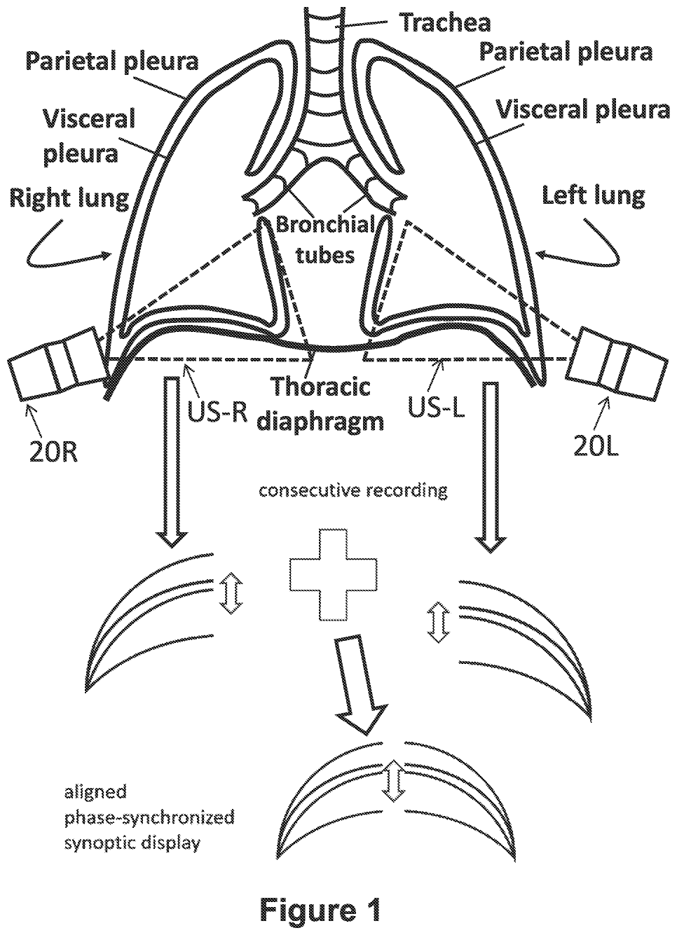

With brief initial reference to FIG. 1, the upper portion thereof diagrammatically shows a simplified representation of the left and right lungs as delineated by the parietal and visceral pleura, the thoracic diaphragm (sometimes referred to herein as the diaphragm for brevity), and contextual anatomy including the trachea and upper portions of the left and right bronchial tubes. For further context, the visceral pleural is adhered to the lungs while the parietal pleura is adhered to the chest wall. As seen, the diaphragm extends underneath (i.e. is positioned anatomically inferior to) the lungs. The thoracic diaphragm is a muscular sheet that extends underneath both left and right lungs. In a healthy patient, during inhalation the diaphragm contracts to expand the lungs downward and thereby draw air into the lungs; during exhalation, the diaphragm relaxes, and the inflated lungs (at least partially) deflate to exhale the air. Hence, the contracting diaphragm thickens during inhalation and is typically at its greatest thickness at end-inspiration, and reduces in thickness during exhalation and is typically at its minimum thickness at end-expiration. By contrast, in the case of a patient who is unable to make any respiratory effort and is receiving mechanical ventilation, the thickening/thinning of the diaphragm over the breathing cycle is reduced or eliminated, although some diaphragm excursion and possibly limited diaphragm thickening/thinning may still occur in response to the mechanical ventilator-driven expansion and contraction of the lungs.

Also shown in the upper anatomical diagram of FIG. 1 are a left-side-positioned ultrasound probe 20L arranged to view the left lung and a corresponding portion of the thoracic diaphragm, and a corresponding right-side-positioned ultrasound probe 20R arranged to view the right lung and a corresponding portion of the thoracic diaphragm. The left and right probe positions 20L and 20R are suitable positions of an external ultrasound probe used for monitoring the diaphragm motion. The illustrated left and right probe positions 20L and 20R are diagrammatic, and in practice various intercostal or subcostal positions may be employed for this purpose. As further illustrated, the left-side-positioned ultrasound probe 20L emits an ultrasound fan-beam US-L that provides for imaging of a portion of the left lung and a corresponding portion of the diaphragm, and in contralateral fashion the right-side-positioned ultrasound probe 20R emits an ultrasound fan-beam US-R that provides for imaging of a portion of the right lung and a corresponding portion of the diaphragm. (In three-dimensional ultrasound imaging, the fan-beams US-L and US-R may be cone-shaped beams). The fan-beams US-L and US-R intersect the diaphragm so as to be able to monitor movement and thickening of the diaphragm in real time. However, as shown typically neither of the fan-beams US-L and US-R penetrate deeply enough into the thorax to simultaneously image both the left and right lungs and corresponding portions of the diaphragm. Consequently, the operator can only observe one lung and corresponding diaphragm portion at any given time. This can be problematic, since observation of small differences between the dynamics of the left and right lungs (and/or corresponding left and right portions of the diaphragm) can be of substantial diagnostic value, for example facilitating diagnosis of an abnormality in one lung based on comparison with the other lung.

As diagrammatically shown in the lower portion of FIG. 1 and as further described below with reference to illustrative embodiments described with reference to the various drawings, embodiments disclosed herein provide an improved visualization by way of combining presentation of a live ultrasound image as a function of time of a second portion of the respiratory system of the patient (for example, the left lung and/or left diaphragm portion of the patient) using the ultrasound probe in the left-position 20L. If the ultrasound imaging device has two ultrasound probes, then one ultrasound probe could be placed in the left-position 20L and the other ultrasound probe in the right-position 20R. In this case, a presentation can be provided of a recorded ultrasound image as a function of time of both a first portion of the respiratory system acquired using the ultrasound probe located in the left-position 20L and a second, contralateral portion of the respiratory system of the patient (in the instant example, the right lung and/or right diaphragm portion of the patient) acquired using the ultrasound probe located in the right-position 20R. Advantageously, both images will be synchronized in time (assuming the ultrasound imaging device timestamps the imaging data acquired by the two ultrasound probes.

However, many ultrasound systems only have a single ultrasound probe, and/or only provide for imaging using a single ultrasound probe at any given time. In this case, there is only one ultrasound probe, and hence imaging at any given time can be done with the probe in the left-position 20L or the right-position 20R, but not both simultaneously. In embodiments disclosed herein that accommodate such “single-probe” ultrasound imaging, a visualization is produced by way of combining presentation of a live ultrasound image as a function of time of a second portion of the respiratory system of the patient (for example, the left lung and/or left diaphragm portion of the patient) using the single ultrasound probe in the left-position 20L, displayed simultaneously and in temporal alignment (by which it is meant respiratory phase alignment, not absolute time alignment; put another way, the temporal alignment is of respiratory cycling of the first ultrasound imaging data and the second ultrasound imaging data) with presentation of a recorded ultrasound image as a function of time of a second, contralateral portion of the respiratory system of the patient (in the instant example, the right lung and/or right diaphragm portion of the patient previously acquired with the single ultrasound probe in the right-position 20R).

To enable this contralateral imaging using a single ultrasound probe, the recorded ultrasound image of the first, contralateral portion of the respiratory system is “pre-recorded,” that is, recorded prior to acquisition of the live ultrasound image. In a variant embodiment, both contralateral portions of the respiratory system are pre-recorded, in which case no live ultrasound image is displayed. As further disclosed herein, the temporal alignment of the simultaneously displayed left and right ultrasound images may utilize various synchronization signals. A suitable synchronization signal is a one-dimensional signal (that is, value samples acquired as a function of time) cycling in correspondence with respiratory cycling of the respiratory system of the patient. By way of nonlimiting illustrative example, the synchronization signal may be a ventilator flow, pressure, volume, or work-of-breathing (WOB) signal recorded by a mechanical ventilator operating to mechanically ventilate the respiratory system of the patient, or a phrenic nerve stimulation (PNS) signal indicative of PNS administered to the patient, or a diaphragm thickness or excursion signal extracted from the first ultrasound imaging data and the second ultrasound imaging data of the respective first and second (i.e. contralateral) portions of the respiratory system.

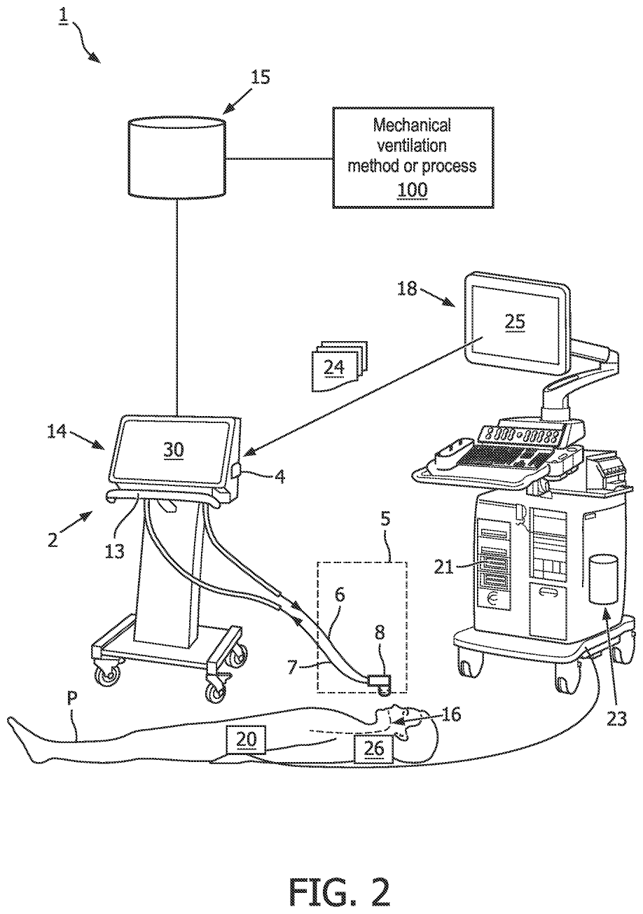

With reference to FIG. 2, a mechanical ventilation or respiration monitoring device 1 is shown. A mechanical ventilator 2 is configured to provide ventilation therapy to an associated patient P is shown. As shown in FIG. 2, the mechanical ventilator 2 includes an outlet 4 connectable with a patient breathing circuit 5 to delivery mechanical ventilation to the patient P. The patient breathing circuit 5 includes typical components for a mechanical ventilator, such as an inlet line 6, an optional outlet line 7 (this may be omitted if the ventilator employs a single-limb patient circuit), a connector or port 8 for connecting with an endotracheal tube (ETT) 16, and one or more breathing sensors (not shown), such as a gas flow meter, a pressure sensor, end-tidal carbon dioxide (etCO₂) sensor, and/or so forth. The mechanical ventilator 2 is designed to deliver air, an air-oxygen mixture, or other breathable gas (supply not shown) to the outlet 4 at a programmed pressure and/or flow rate to ventilate the patient via an ETT. The mechanical ventilator 2 also includes at least one electronic processor or controller 13 (e.g., an electronic processor or a microprocessor), a display device 14, and a non-transitory computer readable medium 15 storing instructions executable by the electronic controller 13.

FIG. 2 diagrammatically illustrates the patient P intubated with an ETT 16 (the lower portion of which is inside the patient P and hence is shown in phantom). The connector or port 8 connects with the ETT 16 to operatively connect the mechanical ventilator 2 to deliver breathable air to the patient P via the ETT 16. The mechanical ventilation provided by the mechanical ventilator 2 via the ETT 16 may be therapeutic for a wide range of conditions, such as various types of pulmonary conditions like emphysema or pneumonia, viral or bacterial infections impacting respiration such as a COVID-19 infection or severe influenza, cardiovascular conditions in which the patient P receives breathable gas enriched with oxygen, or so forth.

FIG. 2 also shows a medical imaging device 18 (also referred to as an image acquisition device, imaging device, and so forth). As primarily described herein, the medical imaging device 18 comprises an ultrasound (US) medical imaging device 18. The illustrative embodiments employ brightness mode (B-mode) ultrasound imaging to assess the diaphragm thickness metric. However, other types of ultrasound imaging or data are contemplated, such as motion mode (M-mode) data collected as a single ultrasound line over a time interval, or so forth.

In a more particular example, the medical imaging device 18 includes an ultrasound probe 20 that is configured to image the lungs and/or diaphragm of the patient P. The US probe 20 is positioned to acquire US imaging data (i.e., two-dimensional (2D) US images) 24 of the diaphragm and/or the lungs of the patient P. For example, the US probe 20 is configured to acquire imaging data of a diaphragm of the patient P, and more particularly 2D US imaging data related to a dimension (e.g., a position, a thickness, and so forth) of the diaphragm of a patient P during inspiration and expiration while the patient P undergoes mechanical ventilation therapy with the mechanical ventilator 2. In another example, the US probe 20 is configured to acquire imaging data of the left lung and the right lung of the patient P during inspiration and expiration, and optionally during inspiration and expiration while the patient P undergoes mechanical ventilation therapy with the mechanical ventilator 2.

In one example, the medical imaging device 18 includes an electronic processor 21 configured to control the ultrasound imaging device 18 to acquire the US images 24, and also includes a non-transitory computer readable medium 23 storing instructions executable by the electronic processor 21. The medical imaging device 18 can also include a display device 25. In another example, the electronic processor 13 of the mechanical ventilator 2 controls the ultrasound imaging device 18 to receive the ultrasound imaging data 24 of the diaphragm of the patient P from the US probe 20. The ultrasound probe 20 allows for continuous and automatic acquisition of the diaphragm thickness data (Tdi) or diaphragm excursion data from the acquired ultrasound imaging data 24.

FIG. 2 also diagrammatically indicates a phrenic nerve stimulation (PNS) device 26 implanted in the patient P. A PNS device 26 is typically an electrode implanted in the patient P and positioned to delivery PNS therapy to a left or right phrenic nerve (i.e., in a neck) of the patient P. The PNS device 26 is implanted at an earlier time during an earlier procedure prior to the mechanical ventilation therapy delivered to the patient P. The PNS device 26 is operatively in communication with the electronic controller 13 of the mechanical ventilator 2 and/or the electronic processor 21 of the ultrasound imaging device 18.

The non-transitory computer readable medium 15 of the mechanical ventilator 2 and/or the non-transitory computer readable medium 23 of the US imaging device 18 stores instructions executable by the electronic controller 13 (and/or the electronic processor 21) to perform a respiration monitoring method or process 100. Although primarily described in terms of the electronic controller 13/non-transitory computer readable medium 15 of the mechanical ventilator 2, the method 100 can similarly be performed by the electronic processor 21/non-transitory computer readable medium 23 of the US imaging device 18.

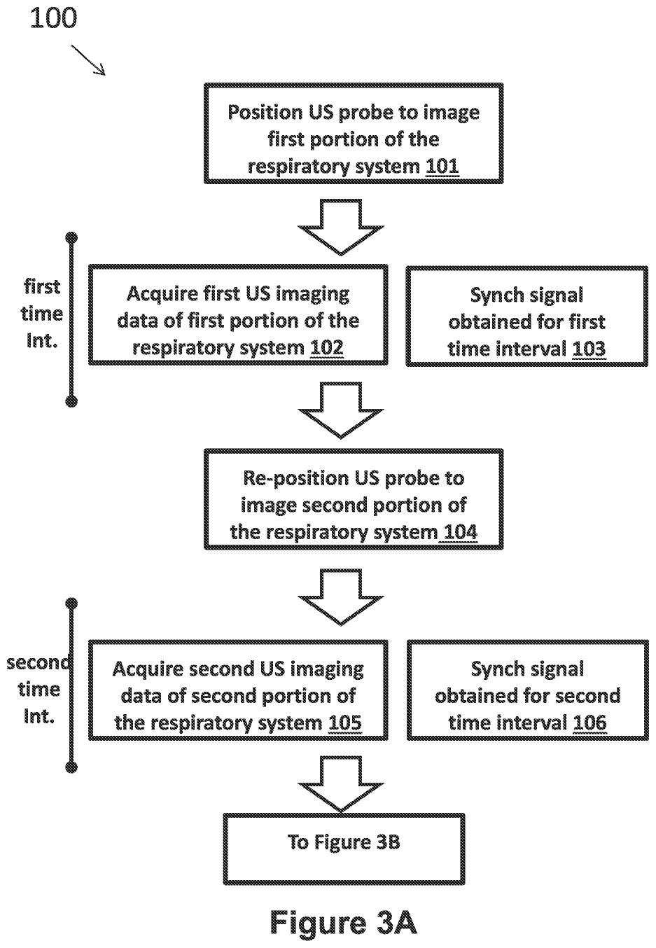

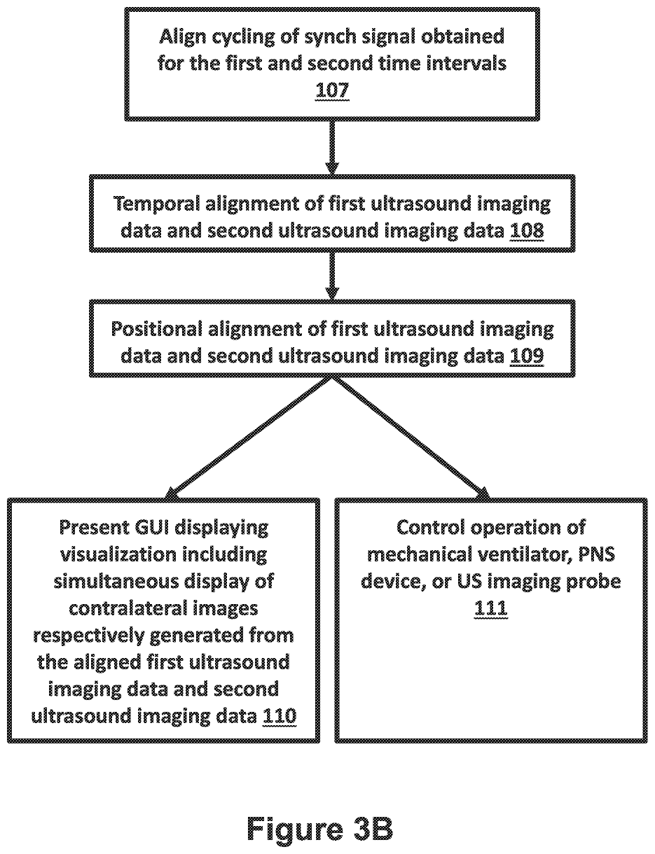

With reference to FIGS. 3A and 3B, and with continuing reference to FIG. 2, an illustrative embodiment of the respiration monitoring method 100 is diagrammatically shown as a flowchart. Although described primarily in terms of the respiratory system of the patient P (e.g., a left lung, a right lung, and a thoracic diaphragm), the method 100 is also applicable to a cardiovascular system of the patient P (i.e., including at least a heart). The example of FIGS. 3A and 3B assumes that there is only the single US probe 20, and/or that the ultrasound imaging device 18 can only acquire images using a single US probe at any given time. (As an example of the latter situation, the US imaging device 18 may be supplied with two or more US probes designed to provide different image characteristics or simply to provide a backup if one US probe fails; but only one of those two or more US probes can be connected with the US imaging device 18 and used to acquire US images at any given time).

As shown in FIG. 3A, To begin the method 100, at an operation 101, the US probe 20 is positioned on the patient P to image a first portion of the respiratory system of the patient P (i.e., a right lung). At an operation 102, first ultrasound imaging data 24 is acquired of the first portion of the respiratory system of the patient over a first time interval, with the ultrasound probe 20 and transmitted to, and received by, the electronic controller 13. The first ultrasound imaging data 24 can be received by the electronic controller 13 as a function of time. In some embodiments, the US imaging data 24 is acquired while the patient P undergoes mechanical ventilation therapy with the mechanical ventilator 2. The US imaging data 24 includes data related to a geometry (e.g., position, thickness, etc.) of the lungs and/or the diaphragm of the patient P. In a particular example, the US imaging data 24 includes a geometry (e.g., position, thickness, etc.) of both the right lung and the left lung of the patient P and include the diaphragm (in particular, movement data of the diaphragm during inhalation and exhalation by the patient P). To do so, the electronic controller 13 can control the ultrasound probe 20 to acquire the ultrasound imaging data 24 and to receive the ultrasound imaging data 24 of the diaphragm of the patient P from the ultrasound probe 20. These images are not necessarily acquired while the patient P is on mechanical ventilation, but instead may be acquired (for example) prior to intubation of the patient.

At an operation 103, a first synchronization signal for the first time interval is obtained. In some examples, the first synchronization signal can be a one-dimensional (1D) signal cycling in correspondence with respiratory cycling of the respiratory system of the patient P.

As further shown in FIG. 3A, the operations 101-103 can be repeated for a second portion of the respiratory system of the patient that is contralateral to the first portion of the respiratory system of the patient (i.e., a left lung). At an operation 104, the US probe 20 is positioned on the patient P to image the second portion of the respiratory system of the patient P. At an operation 105, second ultrasound imaging data 24 is acquired of the second portion of the respiratory system of the patient over a second time interval, with the ultrasound probe 20 and transmitted to, and received by, the electronic controller 13. The second ultrasound imaging data 24 can be received by the electronic controller 13 as a function of time. At an operation 106, a first synchronization signal for the first time interval is obtained. In some examples, the first synchronization signal is can be a 1D signal cycling in correspondence with respiratory cycling of the respiratory system of the patient P.

In some embodiments, the first and/or second synchronization signals can be one or more of a ventilator flow, pressure, volume, or work-of-breathing signal recorded by the mechanical ventilator 2 operating over the first and second time intervals to mechanically ventilate the respiratory system of the patient P; a PNS signal indicative of PNS administered to the patient P by the PNS device 26 over the first and second time intervals; or a diaphragm thickness or excursion signal extracted from the first ultrasound imaging data 24 and the second ultrasound imaging data 24. Although the operations 102 and 104 are described primarily as acquiring at least two regions of the first and second portions of the anatomy, ultrasound imaging data of two, three, four, or more regions can be acquired with the ultrasound probe 20.

With continuing reference to FIGS. 2 and 3A, and referring now to FIG. 3B, at an operation 107, the electronic controller 13 is configured to align the cycling of the first and second synchronization signals. At an operation 108, the first ultrasound imaging data 24 and the second ultrasound imaging data 24 can be aligned, for example, with a temporal alignment by aligning the cycling of the synchronization signal obtained over the first time interval and the cycling of the synchronization signal obtained over the second time interval.

In some embodiments, at an operation 109, the electronic controller 13 is configured to align the first ultrasound imaging data 24 and the second ultrasound imaging data 24 further including positionally aligning the first ultrasound imaging data 24 and the second ultrasound imaging data 24 by aligning images of portions of the thoracic diaphragm represented in the respective first ultrasound imaging data 24 and second ultrasound imaging data 24. To do so, the electronic controller 13 is configured to further includes performing image warping to remove a difference between diaphragm excursion over the respiratory cycling of the respiratory system of the patient P of the portions of the thoracic diaphragm represented in the respective first ultrasound imaging data 24 and second ultrasound imaging data 24.

To align the cycling of the first and second synchronization signals, time-signal curves indicative of respiration data of the patient P over a time of US image acquisition are generated and displayed on the display device 14 of the mechanical ventilator 2 (and/or the display device 25 of the US imaging device 18). To do so, individual one-dimensional (1D) time-signal curves are generated for the US imaging data 24 of a first (i.e., left) lung of the patient P, and a separate time-signal curve is generated for the US imaging data 24 of a second (i.e., right) lung of the patient P. The time-signal curve for the left lung and the time-signal curve for the right lung are synchronized based on corresponding image frames during acquisition of the ultrasound imaging data at a same time for the first lung and the second lung. For example, the image frame for the left lung acquired at t=5 seconds is synchronized with the image frame for the right lung acquired at t=5 seconds. The image frames of the time-signal curves for both lungs are similarly synchronized based on time, and the synchronized time-signal curve is displayed on the display device 14 of the mechanical ventilator 2 (and/or the display device 25 of the US imaging device 18).

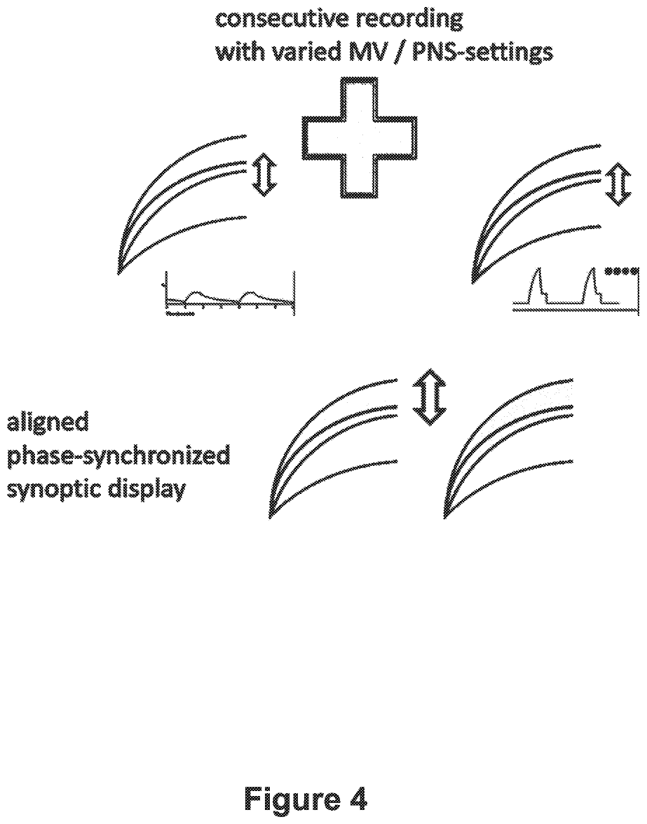

In a particular example, as illustrated in FIG. 4, an aligned and synchronized time-signal curve after subsequent contralateral recordings of the lungs is generated. To do so, a clinician uses the US probe 20 to record one or more cycles on one patient side (i.e., the left lung), then moves the US probe 20 and observes on the opposite patient side (i.e., the right lung). During a subsequent observation, the US imaging data 24 is complemented in a synoptic display, showing two feeds (live & recorded) of two opposing patient sides, in a phase-synchronized fashion as shown in FIG. 3. A live image is paired by the recorded contralateral image of the same respiratory phase selected based on MV treatment data. The clinician can toggle interactively between several available alignment signal curves (one out of the various available time-signal-curves). In addition, the paired contralateral phase-images are aligned vertically for optimized cross correlation in US-beam depth direction. This is achieved by storing with each image frame recorded during the US live feed (stream) the corresponding quantity of the various respiratory time-signal-curves. Then, during synoptic synchronized display, image frames are matched by their closest respiratory phase point. Optionally, for a given time frame of a first series, the best matching phase points A and B of a second series are identified, and an interpolation image between these closest frames is computed, to best match the frame of the first series.

In another example, all previously recorded respiratory cycles (or a live-feed) can be selected to be paired against each other in a synoptic display, i.e., in a phase-synchronized fashion. This allows a direct visual comparison of the effect of various settings on excursion and thickening. The user can toggle interactively between several available time-signal-curves to be used for image alignment. It is desirable that in two graphically juxta-posed US (echo) movies, the diaphragm appears at a similar vertical location in the opposing image frames (which may differ in the two FOVs, respectively). On the other hand, the relative diaphragm excursion in each frame may be desired to be retained. Therefore, a whole respiratory-cycle image-stack S1 (where each 2D echo image frame is considered as one slice of an image stack) is rigidly aligned against a second image-stack S2 by displacement along the x- and y-axes (with a fix z-axis). The optimal alignment shifts can be found by optimizing a e.g., mutual information, correlation, or another image agreement metric. In another embodiment, an object detection model is used (analytical or CNN-based such as YOLO) for explicit tracking of the diaphragm in both time series, and the mutual displacement is found by a regression of the 2D-coordinate curves.

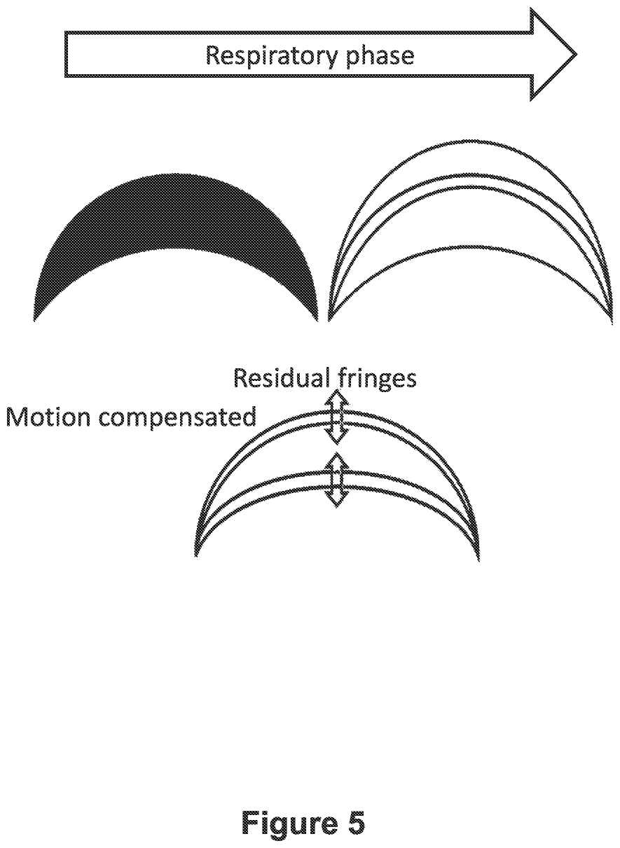

In some embodiments, the electronic controller 13 is configured to further align the first ultrasound imaging data 24 and the second ultrasound imaging data 24 further including performing motion correction to remove at least one uncorrelated motion component of at least one of the first ultrasound imaging data 24 and the second ultrasound imaging data 24. The at least one uncorrelated motion component is not correlated with the cycling of the synchronization signal in correspondence with the respiration cycling of the lungs of the patient P. This is illustrated in FIG. 5. The electronic controller 13 is configured to compensate for a spatially regularized (i.e., in some way uniform) shift along the beam depth, for depth shift patterns which are attributable (i.e., correlated) to the time signal curve from the mechanical ventilator 2. Thus, the effect of the diaphragmatic excursion is removed, while diaphragmatic thickening is remaining as visible motion. The clinician can toggle interactively between several available time-signal-curves to be used for image alignment. This allows an appraisal of different causations for diaphragmatic motion, which, moreover, may differ regionally. Alternatively, the electronic controller 13 compensates for all local image deformation (i.e. not only vertical shifts, but also elastic local warps) which is attributable (i.e., correlated) to the time-signal-curve from the MV (or PNS). All image motions (excursion, thickening, etc.) which are moving in synch with the MV-time signal appear frozen, and residual flutter of the diaphragm lines remains visible in areas which are not governed by the selected phase signal curves. These residuals may reveal spontaneous (untriggered) breathing attempts of the ventilated patient P. For this option, it is desirable to compensate for the diaphragm excursion, while preserving variations in thickness. Therefore, a fully elastic image registration cannot be used. Rather, the medial line of the diaphragm is automatically detected and delineated in each image frame (using analytical or CNN-based semantic segmentation), and the 2D image is deformed (i.e., warped) such as to map the medial line onto a fix straight line or onto the line of a reference image.

For this embodiment, it is desired to filter out any motion in the image which is governed (correlated) by one of the available respiratory phase curves from MV, PNS, etc. Visual appraisal of the residual image movies can thus tell which of the devices appears to be most influential. The decorrelation is be achieved by Independent Component Analysis (ICP), Non-Negative Matrix Factorization (NNMF) (see, e.g., Rabbi, Mohammad & Pizzolato, Claudio & Lloyd, David & Carty, Christopher & Devaprakash, Daniel & Diamond, Laura, Non-negative matrix factorisation is the most appropriate method for extraction of muscle synergies in walking and running. Nature Scientific Reports, 2020), or other algebraic techniques, which decompose the stack of image frames into a function of the phase vector. The phase vector is then set to uniformity, and the image frame are re-composed. In another embodiment, spatio-temporal Generative Models (GAN) or Variational Autoencoders (VAE) are trained to reproduce the observed image frames from one of the available respiratory time-signal-curves. Then the respiratory signal curve is squashed (flattened to uniformity), and virtual image frames are inferenced by the trained model. (This is not an off-site multi-case training, but rather an on-site on-the-fly case-specific training.)

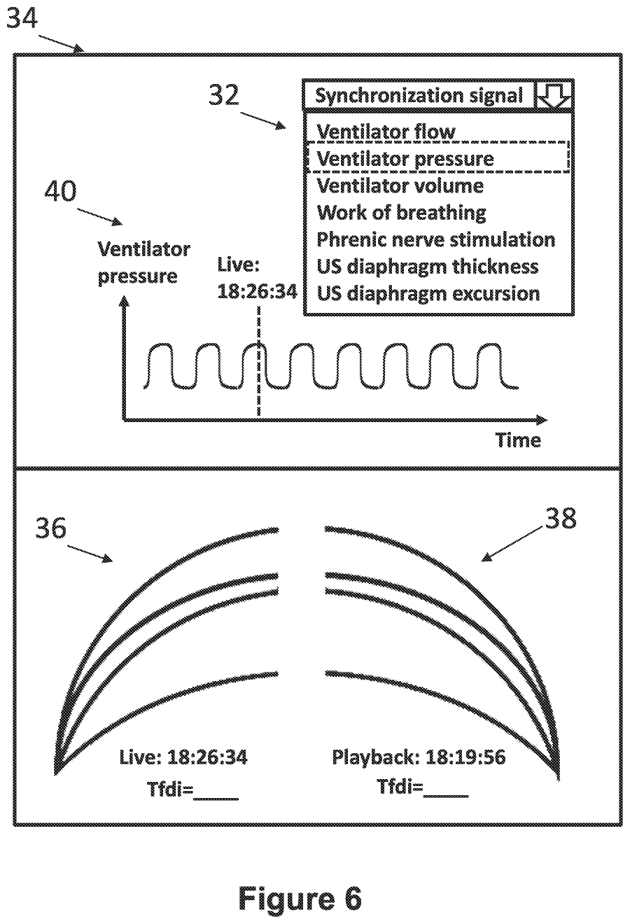

In some embodiments, the electronic controller 13 is configured to obtain a plurality of different signals each of which is a one-dimensional signal cycling in correspondence with respiration cycling of the respiratory system of the patient P, and select the synchronization signal from the obtained plurality of different signals. To do so, in some embodiments, a graphical user interface (GUI) 30 is presented on the display device 14 of the mechanical ventilator 2, and a user dialog 32 (see FIG. 6, described in more detail below) is presented on the GUI 30 by which the synchronization signal can be selected by a clinician. The selection of the synchronization signal is received, via the user dialog 32. In another embodiment, the electronic controller 13 can automatically select the synchronization signal from the plurality of synchronization signals. In some examples, the alignment of the cycling of the synchronization signal and the temporal alignment of the first ultrasound imaging data 24 and the second ultrasound imaging data 24 are updated each time a different selection of the synchronization signal is received via the user dialog 32.

At an operation 110, a visualization 34 of at least a portion of the respiratory system of the patient P. As further shown in FIG. 6, the visualization 34 includes a simultaneous display of an image 36 of the first portion of the respiratory system of the patient and an image 38 of the second portion of the respiratory system of the patient respectively generated from the aligned first ultrasound imaging data 24 and second ultrasound imaging data 24. The user dialog 32 is shown in an upper right portions of the visualization 34 as shown in FIG. 6. In some embodiments (as shown in the lower portion of FIG. 6), the image 36 of the first portion of the respiratory system and the image 38 of the second portion of the respiratory system are cine images respectively generated from the temporally aligned first ultrasound imaging data 24 and second ultrasound imaging data 24.

In some embodiments, the electronic controller 13 is configured to determine a first value of at least one thoracic diaphragm parameter for the first portion of the respiratory system of the patient from the first ultrasound imaging data 24, and determine a second value of the at least one thoracic diaphragm parameter for the second portion of the respiratory system of the patient from the second ultrasound imaging data 24. The visualization 32 further includes display of representations of the first and second values of the at least one thoracic diaphragm parameter, (as shown in the lower portion of FIG. 6). As shown in FIG. 4, the thoracic diaphragm parameter(s) can include one or more of (i) a measure of thoracic diaphragm thickness and/or (ii) a measure of thoracic diaphragm excursion. To do so, the electronic controller 13 is configured to receive treatment data associated with the diaphragm and/or the lungs of the patient P. In one example, the treatment data comprises one or more of (i) mechanical ventilation (MV) data of the patient P while the patient undergoes MV therapy with the mechanical ventilator 2; and/or (ii) PNS data of the patient P while the patient P undergoes PNS therapy with the PNS device 26. The MV data can include, for example, a respiratory pressure of the lungs of the patient P, a flow of air into the lungs of the patient P, a lung volume of the patient P, a works-of-breathing (WoB) time-curve, and so forth. The PNS data can include, for example, an electrical stimulation curve of electrical stimulation supplied by the PNS device 26.

In some embodiments, as show in the upper portion of FIG. 6, the visualization 34 further includes a trendline 40 of the synchronization signal obtained over one or both of the first and second time intervals together with the displayed visualization. The trendline 40 can, in some examples, be a color-coded representation of a magnitude of a correlation between time-signal curves over a time period of a respiratory phase of the patient can be displayed on the display device 14. To do so, the first and second US imaging data 24 (or a recorded moving US image series) is displayed with a spatially resolved color overlay, indicating the magnitude of correlation of the local optical flow with the respiratory phase. A transparent overlay can be used for vanishing correlation (leaving the standard gray-value image), color opacity proportional to local correlation or covariance, color hue signifying the correlation source (MV, PNS, etc.). The clinician may toggle interactively between several available time-signal-curves to be used for image alignment. This allows an appraisal of the different correlation regions, respectively. This is achieved by computing an optical flow for each frame pixel, to extract the flow curve across the frame stack (phase-curve for each pixel), and to cross-correlate this curve against the available time-signal-curves.

In some embodiments, the first time interval is earlier than the second time interval, and the alignment of the cycling of the synchronization signal, the temporal alignment of the first ultrasound imaging data and the second ultrasound imaging data, and the displaying of the visualization 32 are performed in real time during the second time interval. In other embodiments, the alignment of the cycling of the synchronization signal, the temporal alignment of the first ultrasound imaging data and the second ultrasound imaging data, and the displaying of the visualization are performed entirely after completion of both the first time interval and the second time interval.

At an operation 111, operation of a device can be controlled based on the temporally aligned first ultrasound imaging data 24 and second ultrasound imaging data 24. For example, the mechanical ventilator 2 is controlled to adjust one or more parameters of the mechanical ventilation therapy delivered to the patient P based on the temporally aligned first ultrasound imaging data 24 and second ultrasound imaging data 24. In another example, the US imaging probe 20 is controlled to acquire additional imaging data of the diaphragm and lungs of the patient P based on the temporally aligned first ultrasound imaging data 24 and second ultrasound imaging data 24. In a further example, the PNS device 26 is controlled to adjust one or more parameters of the PNS therapy delivered to the patient P based on the temporally aligned first ultrasound imaging data 24 and second ultrasound imaging data 24.

The embodiment of FIGS. 3A and 3B is suitably used when only one US probe 20 is available and/or when only one US probe can be used for imaging at any given time. In another contemplated embodiment (not shown), if two US probes are available then imaging can be simultaneously performed with one US probe positioned at the left-position 20L (see FIG. 1) and the other US probe positioned at the right-position 20R. In this case, the acquired left and right cine images are inherently time-synchronized, and so the acquisition operations 102 and 105 of FIG. 3A can be performed simultaneously (so that the first and second time intervals are the same) and there is no need for the operations 103, 104, 105, 107, and 108. The optional positional alignment 109 is still done in this embodiment, and the visualization 110 is provided as already described.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A respiration monitoring device for monitoring a respiratory system of a patient, the respiratory system comprising a left lung, a right lung, and a thoracic diaphragm of the patient, the respiration monitoring device comprising an electronic controller configured to:
receive first ultrasound imaging data as a function of time acquired of a first portion of the respiratory system of the patient over a first time interval;
receive second ultrasound imaging data as a function of time acquired of a second portion of the respiratory system of the patient over a second time interval wherein the second portion of the respiratory system of the patient is contralateral to the first portion of the respiratory system of the patient; and
present a graphical user interface (GUI) displaying a visualization including simultaneous display of an image of the first portion of the respiratory system of the patient and an image of the second portion of the respiratory system of the patient respectively generated from the first ultrasound imaging data and second ultrasound imaging data.

2. The respiration monitoring device of claim 1 wherein:
the second time interval is different from the first time interval;
the electronic controller is further configured to temporally align respiratory cycling of the first ultrasound imaging data and the second ultrasound imaging data, and
the simultaneous display of the image of the first portion of the respiratory system of the patient and the image of the second portion of the respiratory system of the patient are respectively generated from the temporally aligned first ultrasound imaging data and second ultrasound imaging data.

3. The respiration monitoring device of claim 2, wherein the electronic controller is further configured to:
obtain a synchronization signal for the first time interval and the second time interval wherein the synchronization signal is a one-dimensional signal cycling in correspondence with the respiratory cycling of the respiratory system of the patient; and
align the cycling of the synchronization signal obtained over the first time interval and the cycling of the synchronization signal obtained over the second time interval;
wherein the aligned cycling of the synchronization signal is used to temporally align the respiratory cycling of the first ultrasound imaging data and the second ultrasound imaging data.

4. The respiration monitoring device of claim 3, wherein the synchronization signal is:
a ventilator flow, pressure, volume, or work-of-breathing signal recorded by a mechanical ventilator operating over the first and second time intervals to mechanically ventilate the respiratory system of the patient;
a phrenic nerve stimulation (PNS) signal indicative of PNS administered to the patient over the first and second time intervals; or
a diaphragm thickness or excursion signal extracted from the first ultrasound imaging data and the second ultrasound imaging data.

5. The respiration monitoring device of claim 2, wherein the electronic controller is configured to:
obtain a plurality of different signals each of which is a one-dimensional signal cycling in correspondence with respiration cycling of the respiratory system of the patient; and
select the synchronization signal from the obtained plurality of different signals.

6. The respiration monitoring device of claim 5, wherein the electronic controller is configured to select the synchronization signal from the obtained plurality of different signals by operations including:
presenting a user dialog on the GUI for selecting the synchronization signal from the obtained plurality of different signals; and
receiving the selection of the synchronization signal via the user dialog;
wherein the alignment of the cycling of the synchronization signal and the temporal alignment of the first ultrasound imaging data and the second ultrasound imaging data are updated each time a different selection of the synchronization signal is received via the user dialog.

7. The respiration monitoring device of claim 3, wherein one of:
the first time interval is earlier than the second time interval, and the alignment of the cycling of the synchronization signal, the temporal alignment of the first ultrasound imaging data and the second ultrasound imaging data, and the displaying of the visualization are performed in real time during the second time interval; and
the alignment of the cycling of the synchronization signal, the temporal alignment of the first ultrasound imaging data and the second ultrasound imaging data, and the displaying of the visualization are performed entirely after completion of both the first time interval and the second time interval.

8. The respiration monitoring device of claim 1, wherein, in the displayed visualization:
the image of the first portion of the respiratory system and the image of the second portion of the respiratory system are cine images respectively generated from the first ultrasound imaging data and second ultrasound imaging data; and
the cine image of the second portion of the respiratory system of the patient is positioned contralaterally to the cine image of the first portion of the respiratory system of the patient in correspondence to the contralateral arrangement of the second portion of the respiratory system of the patient to the first portion of the respiratory system of the patient.

9. The respiration monitoring device of claim 1, wherein the electronic controller is further configured to:
determine a first value of at least one thoracic diaphragm parameter for the first portion of the respiratory system of the patient from the first ultrasound imaging data; and
determine a second value of the at least one thoracic diaphragm parameter for the second portion of the respiratory system of the patient from the second ultrasound imaging data;
wherein the visualization further includes display of representations of the first and second values of the at least one thoracic diaphragm parameter.

10. The respiration monitoring device of claim 9, wherein the at least one thoracic diaphragm parameter includes at least one of (i) a measure of thoracic diaphragm thickness and/or (ii) a measure of thoracic diaphragm excursion.

11. The respiration monitoring device of claim 1, wherein the electronic controller is configured to:
positionally align the first ultrasound imaging data and the second ultrasound imaging data by aligning images of portions of the thoracic diaphragm represented in the respective first ultrasound imaging data and second ultrasound imaging data.

12. The respiration monitoring device of claim 11, wherein the positional aligning further includes performing image warping to remove a difference between diaphragm excursion over the respiratory cycling of the respiratory system of the patient of the portions of the thoracic diaphragm represented in the respective first ultrasound imaging data and second ultrasound imaging data.

13. The respiration monitoring device of claim 1, wherein the electronic controller is configured to:
perform motion correction to remove at least one motion component of at least one of the first ultrasound imaging data and the second ultrasound imaging data, wherein the at least one removed motion component is not correlated with respiration cycling of the lungs of the patient.

14. The respiration monitoring device of claim 1, further including at least one of:
an ultrasound imaging device having an ultrasound imaging probe arrangeable respective to the first lung and/or first portion of the thoracic diaphragm of the patient to acquire the first ultrasound imaging data and arrangeable respective to the second lung and/or second portion of the thoracic diaphragm of the patient to acquire the second ultrasound imaging data, wherein the ultrasound imaging probe is controlled to acquire additional imaging data of the diaphragm of the patient based on the temporally aligned first ultrasound imaging data and second ultrasound imaging data;
a mechanical ventilator configured to deliver mechanical ventilation therapy to the patient, wherein receiving the imaging data of a dimension of the lungs of occurs during inspiration and expiration while the patient undergoes mechanical ventilation therapy with the mechanical ventilator; wherein the mechanical ventilator is controlled to adjust one or more parameters of the mechanical ventilation therapy delivered to the patient based on the temporally aligned first ultrasound imaging data and second ultrasound imaging data; and
a phrenic nerve stimulation (PNS) device configured to provide PNS treatment to the patient, wherein the PNS device is controlled to adjust one or more parameters of the PNS therapy delivered to the patient based on the temporally aligned first ultrasound imaging data and second ultrasound imaging data.

15. A respiration monitoring method for monitoring a respiratory system of a patient, the respiratory system comprising a left lung, a right lung, and a thoracic diaphragm of the patient, the respiration monitoring method comprising:
receiving first ultrasound imaging data as a function of time acquired of a first portion of the respiratory system of the patient over a first time interval;
receiving second ultrasound imaging data as a function of time acquired of a second portion of the respiratory system of the patient over a second time interval wherein the second portion of the respiratory system of the patient is contralateral to the first portion of the respiratory system of the patient; and
presenting a graphical user interface (GUI) displaying a visualization including simultaneous display of an image of the first portion of the respiratory system of the patient and an image of the second portion of the respiratory system of the patient respectively generated from the first ultrasound imaging data and second ultrasound imaging data.
